(19) United States
(12) Patent Application Publication
(10) Pub. No.: US 2008/0264859 A1
(43) Pub. Date: Oct. 30, 2008

(54) Method for Initiating Microbiological Processes in Artificial Waters

(75) Inventor: Thomas Willuweit, Hof (DE)

Correspondence Address: GUDRUN E. HUCKETT DRAUDT, SCHUBERTSTR. 15A, WUPPERTAL, 42289, DE

(73) Assignee: Soll GmbH, Hof (DE)

(21) Appl. No.: 12/089,210

(22) PCT Filed: Oct. 6, 2006
(86) PCT No.: PCT/DE2006/001760

§ 371 Date: Apr. 4, 2008

(30) Foreign Application Priority Data
Oct. 7, 2005 (DE) 10 2005 048 461.1

(51) Int. Cl. C02F 3/34; C12N 11/00; C02F 3/00

(52) U.S. Cl. 210/615; 435/174; 210/601

(57) ABSTRACT

In a method for initiating microbiological processes in stagnant and flowing bodies of water heterotrophic microorganisms in immobilized form are added to the water. The heterotrophic microorganisms are selected from heterotrophic nitrifying microorganisms. The heterotrophic microorganisms are present in dried form and are immobilized on a material that is suitable as a water filter or can be pressed into a tablet.

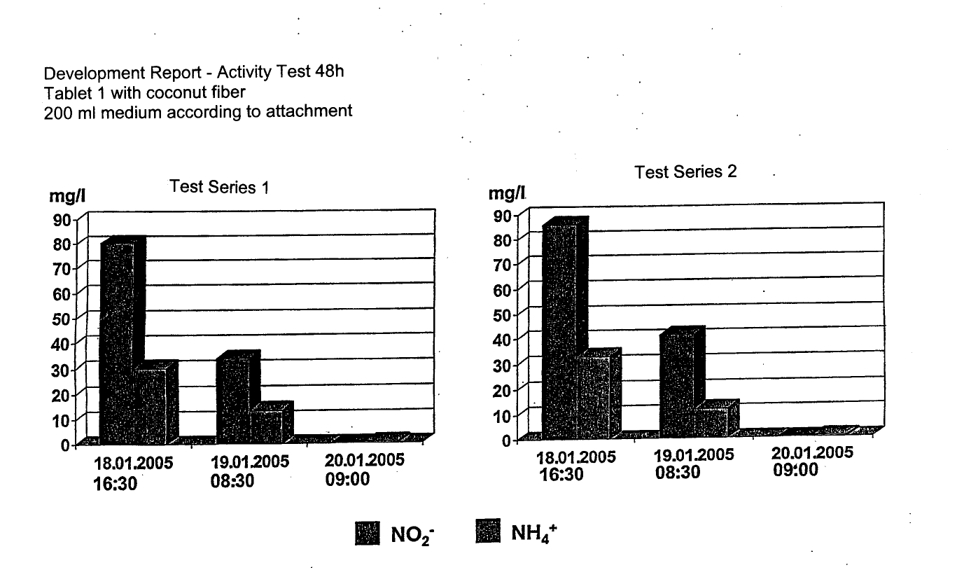

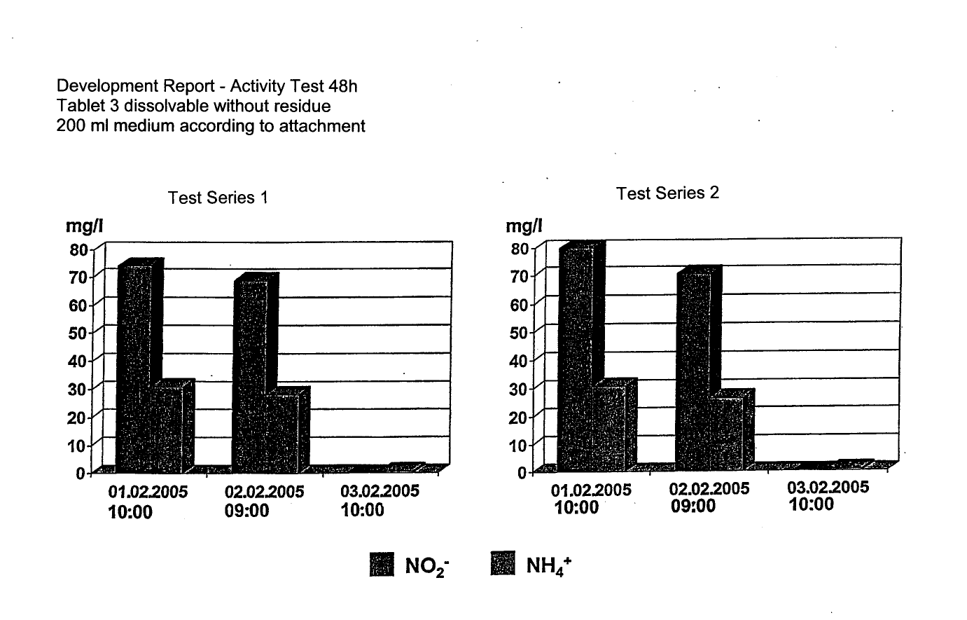

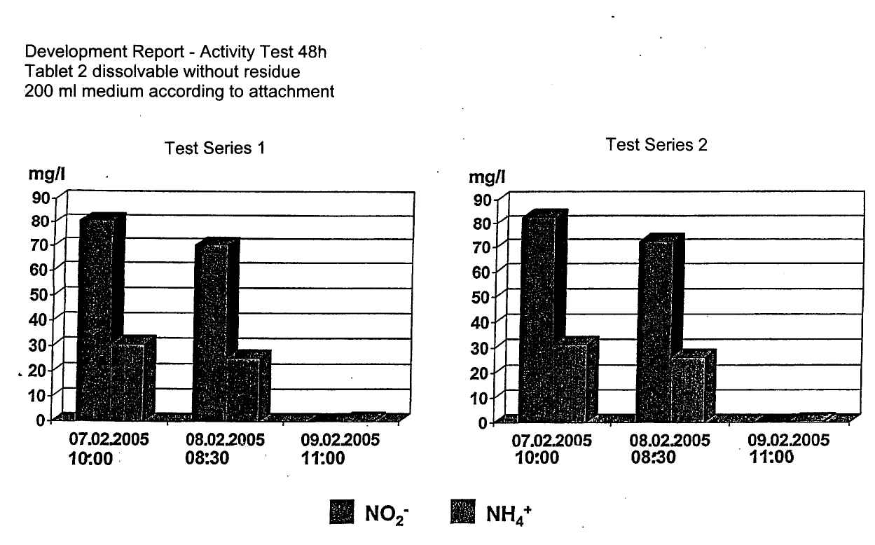

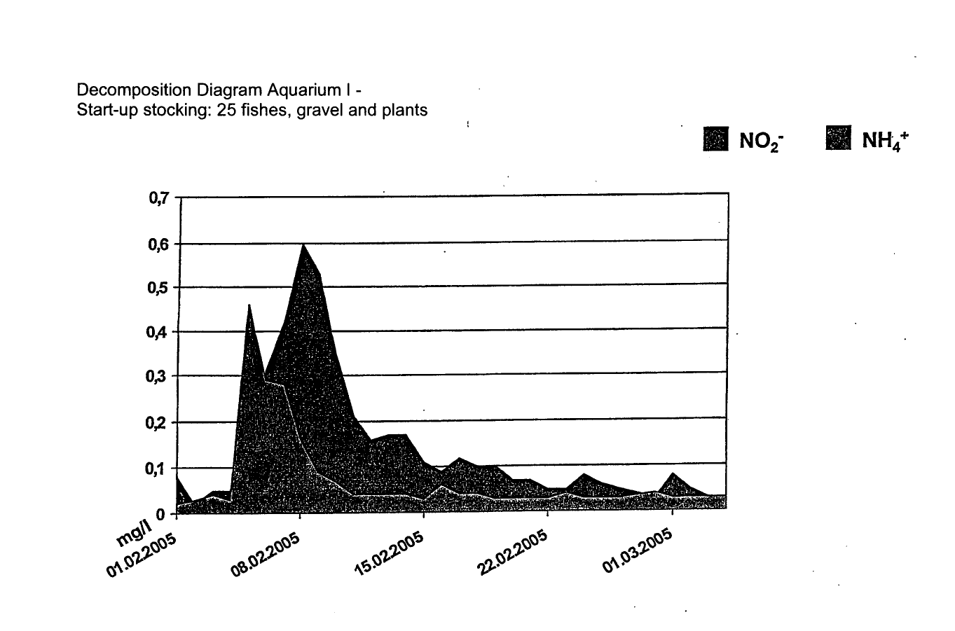

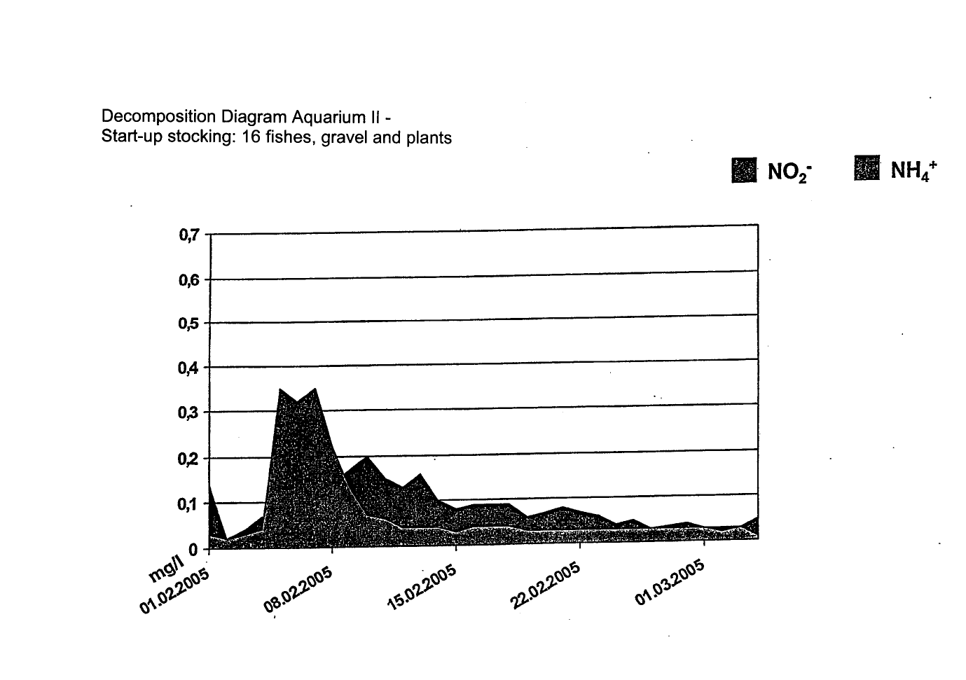

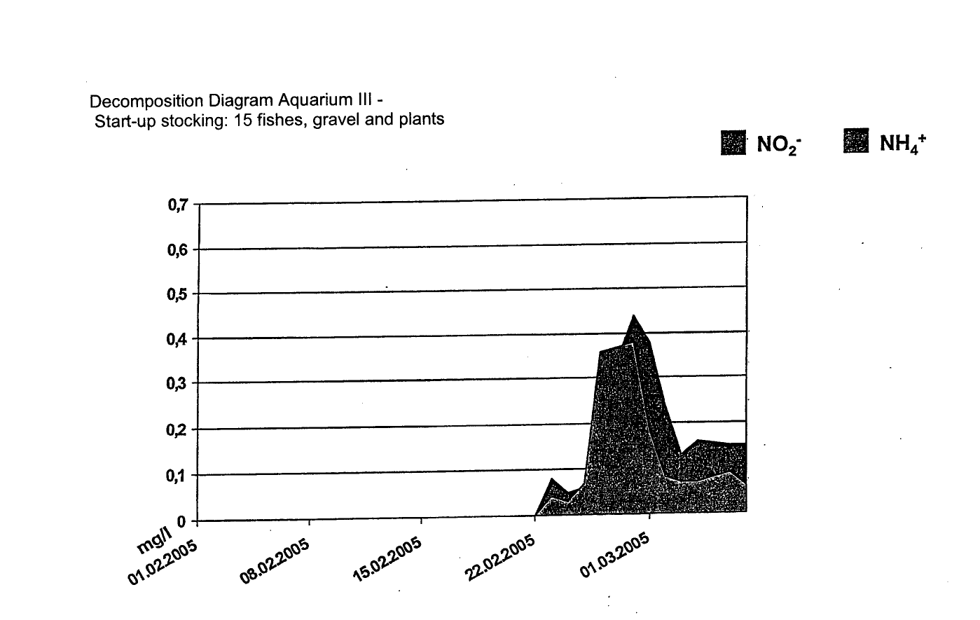

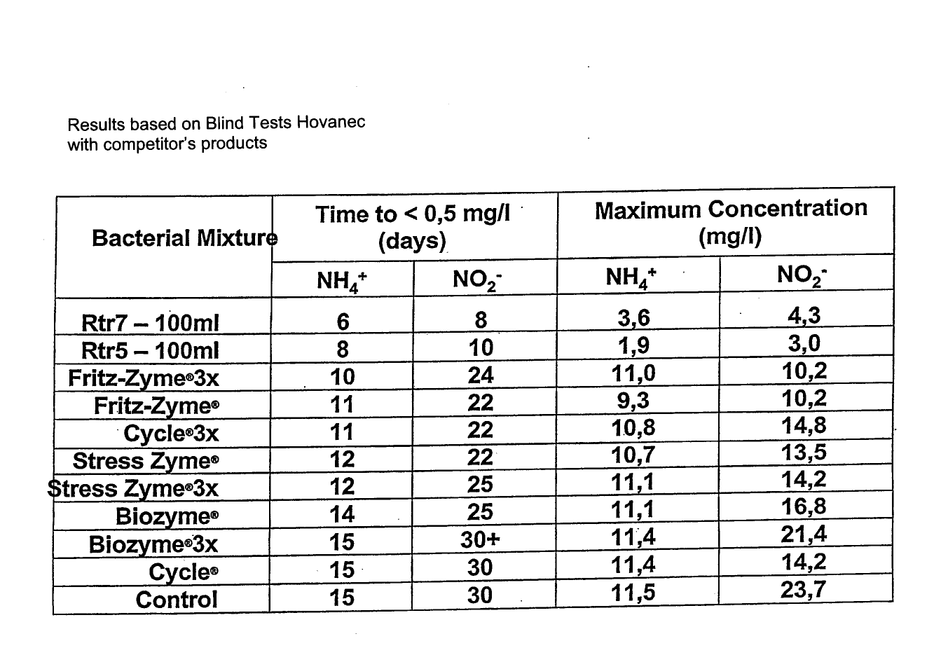

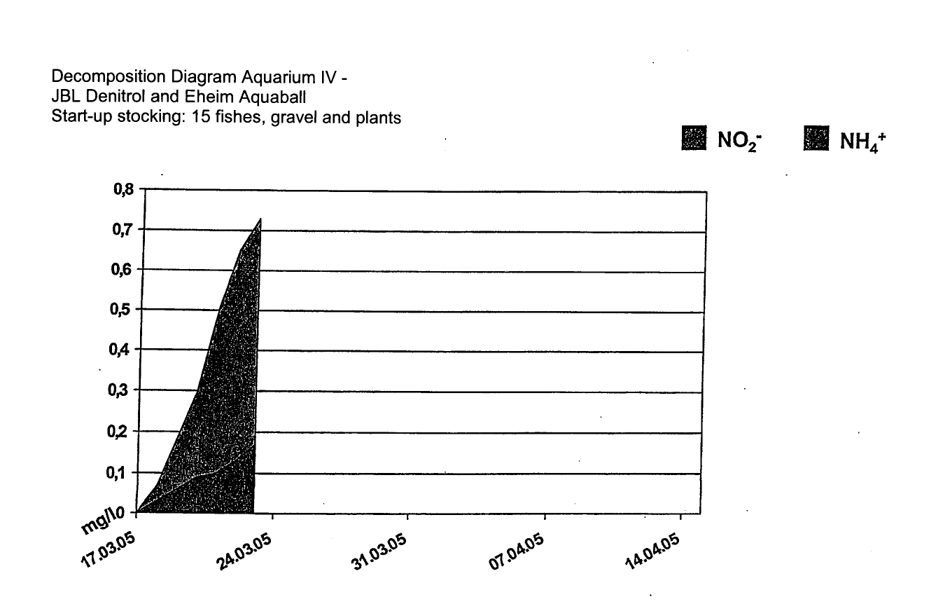

The present invention relates to a method for initiating microbiological processes in stagnant and flowing bodies of water, in particular, in aquariums and/or garden ponds, in which the process is initiated by addition of heterotrophic microorganisms, as well as to a kit of parts, comprising immobilized microorganisms as well as a receptacle for the immobilized microorganisms that can be introduced into stagnant and/or flowing bodies of water.

Stagnant and flowing natural bodies of water in general have a certain self-purification power, i.e., pollutants can be decomposed to a limited extent. These self-purification powers however are insufficient in case of greatly loaded bodies of water. To these bodies of water microorganisms are often added for assisting the (self)purification process which microorganisms in particular can decompose harmful nitrogen compounds to innocuous compounds such as elementary nitrogen.

Aquariums and garden ponds as well as other newly installed artificial bodies of water experience at start-up the problem that between the colonized living beings including introduced fishes and aquatic plants there is no equilibrium and such equilibrium must be established first. Upon start-up ammonia is produced first which dissolves in water so as to form ammonium ions and accumulates in the water, i.e., the ammonia contents first passes through a maximum. During the further development, ammonia oxidants (autotrophic bacteria) will colonize and convert the existing ammonia to nitrite. Based on this, in a time period of 2 to 20 days after the occurrence of the ammonia maximum a maximum of nitrite occurs in the water.

The formation of nitrite leads to colonization of nitrite-oxidizing autotrophic bacteria that convert nitrite to nitrate so that the nitrite concentration drops while at the same time the nitrate concentration rises.

Ammonia (ammonium) as well as nitrite are poisonous to fish, i.e., already at the beginning of the start-up of the newly installed artificial body of water the fishes held therein are exposed to relatively high concentrations of ammonia and nitrate so that their quality of life is greatly diminished. The observed maxima of ammonia and nitrite, depending on how many plants and fishes are contained in the body of water (aquarium or pond), are at 4 to 30 mg/l nitrite and 1 to 8 mg/l ammonium. The greatest danger when newly installing artificial bodies of water and also when newly starting an aquarium is that the plants and fishes living therein are damaged by the generated contents of ammonia and nitrite and, in the worst case, will die off. In the field of aquaristics already at minimal amounts of dissolved nitrogen compounds problems will occur because, for example, ammonia is toxic to fish already at a concentration of 0.01 mg/l. In particular in aquariums that represent artificial systems the self-purification process is particularly interference-sensitive already at minimal quantities of contained foreign substances such as nitrogen compounds.

The prior art discloses values between 10 and 30 mg nitrate per liter as a nitrite maximum after 10 to 15 days. A nitrite maximum between 10 and 15 mg nitrite per liter is described in Köhler, Diskus-Brief 06/1997.

WO 02/24583 discloses microbiological cultures for initiation of decomposition processes in bodies of water which culture contains substantially autotrophic microorganisms. However, autotrophic microorganisms have the disadvantage that they cannot be converted into a storage-stable dry form.

The present invention has the object to provide a method that makes it possible to locally purify or process stagnant and/or flowing bodies of water by using microorganisms, i.e., to decompose harmful substances by employing microorganisms. A further object is to provide a composition with which the usually occurring nitrite peak can be cut in half by means of the bacteria starter product wherein the biomass that is used as a starter should be available as a permanently nonperishable product and, under conditions of use, decompose undesirable components such as ammonium and nitrite.

Object of the present invention is therefore a method for initiating microbiological processes in stagnant and flowing bodies of water, in particular in aquariums and/or garden ponds, in which the process is initiated by adding heterotrophic microorganisms, characterized in that the microorganisms are added in immobilized form.

The microorganisms according to the invention are preferably heterotrophic microorganisms. They are characterized in that they are stable as a dried product over a long period of time and can develop their activity again after contact with water.

Suitable microorganisms are in particular Bacillus azotoformans, DSM number 1046 (denitrifying bacterium); Sporosarcina psychrophila, DSM number 3 (denitrifying bacterium); Arthrobacter globiformis, DSM number 20124 (assimilates nitrate or nitrite under aerobic conditions); Pseudomonas fluorescens, DSM number 11387 (nitrilase generation/nitrogen oxidation); Pseudomonas putida, DSM number 11388 (nitrilase generation/nitrogen oxidation); Sulforospirillum delayianum, DSM number 6946 (nitrite reductase). The microorganisms can be used alone or as a discretionary mixture.

The heterotrophic microorganisms are preferably selected from heterotrophic nitrifying microorganisms, such as denitrifying microorganisms, in particular Bacillus azotoformans and Sporosarcina psychrophila; as well as Arthrobacter globiformis; Pseudomonas fluorescens; Pseudomonas putida; and/or Sulforospirillum delayianum.

In an especially preferred embodiment, the microorganisms are present in dried form. In this form, they can be stored over a long period of time which is advantageous in particular when the microorganisms are to be distributed through commercial channels. The dried microorganisms can be packaged in ways known in the art.

It has been found to be particularly advantageous to immobilize the microorganisms on or in a suitable material. By immobilization the microorganisms can be processed directly into a form suitable for retail. Suitable materials are in particular those that in water or bodies of water can be used also as water filters or as a part thereof.

The microorganisms can be applied first onto suitable materials and subsequently can be further processed, for example, by application onto or processing to water filters or in that the materials loaded with microorganisms are processed to tablets and packaged in appropriate bags.

Examples of suitable materials to which the microorganisms can be applied are those that can serve as filter materials, i.e., have a large surface area. They include natural and synthetic sponges, fibers, foams, porous glasses or ceramics or sintered materials, paper, porous plastic materials. In this way, they can be introduced into the water together with the water filters that are usually employed in artificial bodies of water, including aquariums, so that additional devices etc. are avoided in such bodies of water. The immobilized microorganisms can also be applied, inasmuch as they are present in a finely dispersed form, onto the material from which the water filter is to be produced. In a further embodiment, the microorganisms are applied directly onto the filter material. The material for the water filter can be selected from any suitable material, for example, natural and synthetic polymer foams, water-permeable materials based on cellulose, porous materials with large surface area based on water-insoluble inorganic materials such as silicates.

In a further embodiment, the microorganisms are processed in the form of a tablet that is partially or entirely dissolvable in water. For this purpose, they are applied onto materials and subsequently compressed, for example, pressed to tablets. The preferably dried microorganisms can also be portioned into bags. These bags can also be water-soluble, i.e., can be directly introduced into water.

When the microorganisms are used in the form of tablets, the tablets are added to the water. The tablet decomposes or completely dissolves. The immobilized microorganisms are reactivated by contact with water and colonize in the water so that the microbiological process is initiated. Even when the microorganisms are not applied to a component that is fixedly installed in the water, for example, a water filter or a part thereof, the microorganisms are able to initiate the appropriate decomposition processes.

A particularly effective start of the microbiological processes can be achieved when the microorganisms are used in combination with a reducing agent. As a reducing agent those are preferred that have no negative effect on the water quality and that do not impair the activity of microorganisms, plants, and fishes contained in the water. Derivatives of sulfinic acid, for example, sodium hydroxy methane sulfinate and its hydrates, are well suited. The reducing agent is preferably not added simultaneously with the heterotrophic microorganisms but delayed. A suitable point in time has been determined to be 6 to 11 days after starting the microbiological process.

A further object concerns means for initiating microbiological processes in stagnant and flowing bodies of water, in particular in aquariums and/or garden ponds, comprising heterotrophic microorganisms in immobilized form and a reducing agent.

In order to be able to add the microorganisms and the reducing agent separately to the water, they are present in separate form. In one possible embodiment, the microorganisms are present as described above in dried form, for example, applied onto a suitable material such as a filter material, or processed in tablet form or in form of portioned bags. The reducing agent can also be present in the form of tablets or in the form of portioned bags wherein the portioned bags can also be water soluble, i.e., can be added directly to the water.

The reducing agent that is separate and added preferably delayed is suitably present also in compressed form such as a tablet. The tablet form has not only advantages in regard to storage and transport but is also in particular user-friendly because handling is easy. Since the concentration of reducing agent in the compressed form is in certain situations too high, it can be diluted with conventional substances that do not impair the water quantity. Examples of such substances are alkali and earth alkali carbonates and hydrogen carbonates, alkali and earth alkali sulfates, alkali and earth alkali chlorides etc. wherein sodium carbonate or sodium hydrogen carbonate are preferred.

A further object of the present invention is a kit of parts comprising immobilized microorganisms as well as a receptacle for the immobilized microorganisms. This kit of parts can be introduced directly into stagnant and/or flowing bodies of water, for example, as filter material of the water filter or as a water filter.

### EXAMPLES

So-called aquarium startups with a daily amount of food of 300 mg were performed for which a nitrite peak under 0.3 mg/l was obtained.

The blind values of these tests showed between 4.6 and 11.2 mg nitrite per liter. In the blind tests nitrite was observed for 6 days above 2 mg nitrite per liter!

Since as a result of the composition of the tablets a pH range of 7.6-8.0 is stabilized, based on these values no fish mortality is to be expected (see supra) and was also not observed in the tests (with the exception of individual fishes).

Test Series:

1)

The tests with coconut fiber tablets showed excellent results; the daily amount of food was not documented.

2)

The tests with a tablet that dissolves completely and minimal amount of food (approximately 70 mg/d) showed excellent results (all values below 1 mg/l). As a filler, bicarbonate was added to the tablet; this leads to a pH stabilization at pH 7.6-8.0 in all aquariums.

3) Sodium Hydroxy Methane Sulfate Dihydrate

The tablet was produced with this additive in order to introduce optionally from the beginning a nitrite reducing potential.

The tests with this tablet were clearly negative; high nitrite peaks occurred, even though with delay, in all aquariums Two of the tests were stopped, in two tests the active ingredient was supplemented.

Both supplemented aquariums reacted by immediate nitrite reduction and subsequent fast decomposition.

5)

Tests with higher and very high amounts of food were initiated with one tablet and two tablets without additive. The aquariums with one tablet showed better results. Between the 8th and the 10th day a nitrite increase was observed; however it was lower in comparison to the above cited values and the blind tests (see supra). The addition of the 2nd tablet with the additive sulfinate on the 8th to 10th day significantly lowers the nitrite concentration immediately and sustained on a thirty-day scale.

The addition of the additive does not lower the ammonium concentration but the additive prevents however the measurability of ammonium by means of test kits and photometric methods. (Ammonium is masked.)

The combination with a 2nd tablet presents an excellent solution.

It was achieved that in all tests the nitrite concentration reached levels above 0.6 mg nitrite per liter only on one day. In the worst case the 1-day peak reached 1.9 mg nitrate per liter. These tests were performed with very high quantities of food (300 mg/d).

No increased mortality was observed among the aquarium startups. Mostly, the fishes survived the tests well (no total loss, no mass mortality).

The effectivity is apparent from all tests with occupied aquariums.

What is claimed is:

1.-11. (canceled)

12. A method for initiating microbiological processes in stagnant and flowing bodies of water, the method comprising the step of adding heterotrophic microorganisms in immobilized form.

13. The method according to claim 12, wherein the heterotrophic microorganisms are selected from heterotrophic nitrifying microorganisms.

14. The method according to claim 13, wherein the heterotrophic nitrifying microorganisms are selected from the group consisting of Bacillus azotoformans; Sporosarcina psychrophila; Arthrobacter globiformis; Pseudomonas fluorescens; Pseudomonas putida; and Sulforospirillum delayianum.

14. (canceled)

15. The method according to claim 12, wherein the microorganisms are immobilized on or in a suitable material, wherein the suitable material can serve as a water filter or as a part of a water filter.

16. The method according to claim 15, wherein the suitable material serving as a water filter is selected from natural and synthetic polymer foams, water-permeable materials based on cellulose, porous materials with large surface area based on water-insoluble inorganic materials, sponges, fibers, foams, porous glasses or ceramics or sintered materials, paper, plastics.

17. The method according to claim 16, wherein the water-insoluble inorganic materials are silicates.

18. The method according to claim 12, wherein the microorganisms are immobilized on or in a suitable material and processed in the form of a tablet that is entirely or partially dissolvable in the water.

19. The method according to claim 12, further comprising the step of adding a reducing agent in the start-up phase of the microbiological process.

20. The method according to claim 19, wherein the reducing agent is selected from derivatives of sulfinic acid.

21. The method according to claim 19, wherein the reducing agent is added 6 to 11 days after starting the microbiological process.

22. An agent for initiating microbiological processes in stagnant and flowing bodies of water, the agent comprising heterotrophic microorganisms in immobilized form and a reducing agent.

23. The agent according to claim 22, wherein the microorganisms and the reducing agent are present in separate form.

24. A kit comprising immobilized microorganisms and a receptacle for the immobilized microorganisms.

25. The method according to claim 12, wherein the heterotrophic microorganisms are present in dried form.
